(19) United States
(12) Patent Application Publication
(10) Pub. No.: US 2024/0197934 A1
(43) Pub. Date: Jun. 20, 2024

(54) ANTIMICROBIAL SURFACE BASED ON ELECTRIC FIELD TREATMENT

(71) Applicant: Georgia Tech Research Corporation, Atlanta, GA (US)

(72) Inventors: Xing Xie, Atlanta, GA (US); Feifei Liu, Atlanta, GA (US); Ting Wang, Atlanta, GA (US)

(21) Appl. No.: 18/540,223

(22) Filed: Dec. 14, 2023

Related U.S. Application Data
(60) Provisional application No. 63/387,399, filed on Dec. 14, 2022.

(51) Int. Cl. A61L 2/03; A61L 2/24; A61L 2/26

(52) U.S. Cl. CPC A61L 2/03; A61L 2/24; A61L 2/26; A61L 2202/14

(57) ABSTRACT

An exemplary embodiment of the present disclosure provides a system for microorganism and/or biofilm inactivation that can comprise an antimicrobial surface. The antimicrobial surface can comprise a plurality of electrodes arranged in a predetermined pattern. The antimicrobial surface can include an insulative material which can coat at least a portion of each of the plurality of electrodes. The antimicrobial surface can include an external power source that can be configured to supply electrical power to the plurality of electrodes to at least in part induce the electric field via the plurality of electrodes.

### CROSS-REFERENCE TO RELATED APPLICATIONS

This application claims the benefit of U.S. Provisional Application Ser. No. 63/387,399, filed on 14 Dec. 2022, which is incorporated herein by reference in its entirety as if fully set forth below.

### GOVERNMENT LICENSING RIGHTS

This invention was made with government support under Grant No. GR1845354, awarded by the National Science Foundation (NSF). The government has certain rights in the invention.

### FIELD OF THE DISCLOSURE

The various embodiments of the present disclosure relate generally to systems and methods of inactivating and/or inhibiting bacteria accumulation on surfaces, and more specifically inactivating and/or inhibiting bacteria accumulation on surfaces utilizing interdigitated and/or nano wedge electrodes.

### BACKGROUND

Unwanted bacteria growth can cause serious problems and raise significant public health concerns. For instance, unwanted bacteria growth may form biofilms on surfaces like medical devices, equipment in food processing or other industries, and city infrastructures like water distribution systems. In the case of city infrastructures, such as water distribution systems, biofilms can cause problems such as clogging and biocorrosion, thus leading to significant financial and health impacts. The presence of biofilms in water distribution systems can serve as reservoirs for pathogens and antibiotic resistant genes, which can continuously interact with the delivered water. Resultantly, biofouling on membrane modules in industries, such as medical devices and instruments, food processing equipment, city infrastructure equipment, and the like, can cause process failure, material waste, or even safety issues. Accordingly, biofilm control and antifouling methods are crucial and highly demanded.

Current conventional biofilm control methods, such as mechanical cleaning, chemical sterilization, and UV light disinfection, are inefficient and create intensive energy and fiduciary costs. Some current conventional solutions for biofilm control attempt to couple applied electric fields to surfaces via electrostatic repulsion for microbe adhesion mitigation. The drawback of this technology, however, is that electrostatic repulsion for microbe adhesion mitigation is not a sustainable and long-lasting solution for anti-biofouling or biofilm control performance on surfaces. Another current conventional biofilm control method is using electrochemical reactions for bacteria inactivation or biofilm agitation. However, the process of creating the electrochemical reactions may induce bubbles, generate unwanted byproducts, or cause safety issues when utilized. Some current conventional biofilm control methods include employing principles of electroporation, which creates pores in the membrane of bacterium upon application of a strong magnetic field. However, the efficacy of electroporation is directly proportional to the strength of the electric field generated, thus requiring high energy input which can cause safety concerns.

Thus, a need exists for systems and methods of biofilm control and mitigation that can consistently and efficiently promote antimicrobial and antifouling surfaces on demand for real-life applications.

### BRIEF SUMMARY

An exemplary embodiment of the present disclosure provides a system for microorganism and/or biofilm inactivation that can comprise an antimicrobial surface. The antimicrobial surface can comprise a plurality of electrodes arranged in a predetermined pattern. The antimicrobial surface can include an insulative coating material which can coat at least a portion of each of the plurality of electrodes. The antimicrobial surface can include an external power source that can be configured to supply electrical power to the plurality of electrodes to at least in part induce the electric field via the plurality of electrodes.

In any of the embodiments disclosed herein, the antimicrobial surface can be flexible and can be configured to be affixed to flat and curved surfaces.

In any of the embodiments disclosed herein, the antimicrobial surface can further comprise one or more contact pads affixed to the plurality of electrodes. The one or more contact pads can be configured to be electrically connected to the external power source.

In any of the embodiments disclosed herein, the predetermined pattern of the plurality of electrodes can be an interdigitated pattern with each of the plurality of electrodes separated by a horizontal spacing interval of at least approximately 10 nanometers (nm).

In any of the embodiments disclosed herein, the predetermined pattern of the plurality of electrodes can be an interdigitated pattern with a horizontal spacing interval between the one or more contact pads of at least approximately 10 micrometers (μm).

In any of the embodiments disclosed herein, the electric field generated by the plurality of electrodes can be at least approximately 1 kilovolt per centimeter (1 kV/cm).

In any of the embodiments disclosed herein, the system can further comprise one or more nanowedges distributed between the plurality of electrodes. Each of the one or more nanowedges can have a predetermined horizontal or vertical spacing interval, with respect to each other.

In any of the embodiments disclosed herein, the one or more nanowedges can be affixed to the plurality of electrodes, each of the one or more nanowedges having the predetermined horizontal or vertical spacing interval, with respect to each other.

In any of the embodiments disclosed herein, the insulative material can be configured to electrically protect the plurality of electrodes against short circuiting. The insulative material can comprise a material selected from the group consisting of polymers and metal oxides.

In any of the embodiments disclosed herein, the external power source can be further configured to provide alternating current (AC) electrical power to the plurality of electrodes to induce the electrical field.

In any of the embodiments disclosed herein, the external power source can be configured to provide AC electrical power with a voltage range of 1-500 volts (V) and a corresponding frequency range of 10⁻³-10⁹ Hertz (Hz).

In any of the embodiments disclosed herein, the external power source can be configured to provide AC electrical power with a waveform selected from a group consisting of: sinusoidal, exponential, triangle, square, and bell.

In any of the embodiments disclosed herein, the external power source can be further configured to provide direct current (DC) electrical power to the plurality of electrodes to induce the electrical field.

In any of the embodiments disclosed herein, the external power source can be configured to provide DC electrical power with a voltage range between 1-500 V.

In any of the embodiments disclosed herein, the external power source can be configured to provide DC electrical power periodically in alternating polarities.

In any of the embodiments disclosed herein, the external power source can be configured to provide DC electrical power with one or more predetermined pulse widths.

Another exemplary embodiment of the present disclosure provides a system that can comprise a substrate, a plurality of electrodes that can be distributed on the substrate, and a power source that can be configured to provide power to the plurality of electrodes such that the plurality of electrodes can produce an electric field on at least a portion of the substrate sufficient to achieve an antimicrobial or antifouling result.

In any of the embodiments disclosed herein, the system can further comprise a plurality of nanowedges distributed between the plurality of electrodes. Each of the plurality of nanowedges can have an aspect ratio between 10 and 1,000,000.

In any of the embodiments disclosed herein, the plurality of electrodes can be interdigitated.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

The patent or application file contains at least one drawing executed in color. Copies of this patent or patent application publication with color drawing(s) will be provided by the Office upon request and payment of the necessary fee.

FIG. 1A is a plot of simulation results for an exemplary antimicrobial surface for an antimicrobial/antifouling system, in accordance with an exemplary embodiment of the present disclosure.

FIG. 1B is a view of a side-by-side comparison between a scanning electron microscope (SEM) image and an optical microscopy image of a microorganism inactivated by the antimicrobial/antifouling system, in accordance with an exemplary embodiment of the present disclosure.

FIG. 2A is a microscopy view of a predetermined electrode pattern connected to one or more contact pads of an antimicrobial/antifouling system, in accordance with an exemplary embodiment of the present disclosure.

FIG. 2B is an alternate microscopy view of a predetermined electrode pattern connected to one or more contact pads of an antimicrobial/antifouling system, in accordance with an exemplary embodiment of the present disclosure.

FIG. 2C is an illustration of design parameters for a predetermined electrode pattern connected to one or more contact pads of an antimicrobial/antifouling system, in accordance with an exemplary embodiment of the present disclosure.

FIG. 2D is an illustration of alternate structures for a predetermined electrode pattern as shown in FIG. 2C of an antimicrobial/antifouling system, in accordance with an exemplary embodiment of the present disclosure.

FIG. 3 is a microscopy view of a plurality of nanowedges between a plurality of electrodes with an inlet of one of the plurality of nanowedges of an antimicrobial/antifouling system, in accordance with an exemplary embodiment of the present disclosure.

FIG. 4A is a plot of simulation results for a plurality of nanowedges having a horizontal interval of spacing of approximately 0.5 μm between each of the plurality of nanowedges, in accordance with an exemplary embodiment of the present disclosure.

FIG. 4B is a plot of simulation results for a plurality of nanowedges having a horizontal interval of spacing of approximately 16 μm between each of the plurality of nanowedges, in accordance with an exemplary embodiment of the present disclosure.

FIG. 4C is a plot of the relationship between the strength of an applied electric field versus horizontal intervals of spacing between each of the plurality of nanowedges, in accordance with an exemplary embodiment of the present disclosure.

FIG. 5A is a plot showing the relationship between applied nanosecond pulses from an external power source and antimicrobial efficiency of current conventional antimicrobial/antifouling technologies, in accordance with an exemplary embodiment of the present disclosure.

FIG. 5B is a plot showing the plot showing the relationship between effective treatment time and antimicrobial efficiency of current conventional antimicrobial/antifouling technologies, in accordance with an exemplary embodiment of the present disclosure.

FIG. 6A is a plot showing the relationship between applied nanosecond pulses from an external power source and antimicrobial efficiency of an exemplary embodiment of the antimicrobial/antifouling system, in accordance with an exemplary embodiment of the present disclosure.

FIG. 6B is a plot showing the relationship between effective treatment time and antimicrobial efficiency of an exemplary embodiment of the antimicrobial/antifouling system, in accordance with an exemplary embodiment of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

By “comprising” or “containing” or “including” is meant that at least the named compound, element, particle, or method step is present in the composition or article or method, but does not exclude the presence of other compounds, materials, particles, method steps, even if other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described as making up the various elements of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

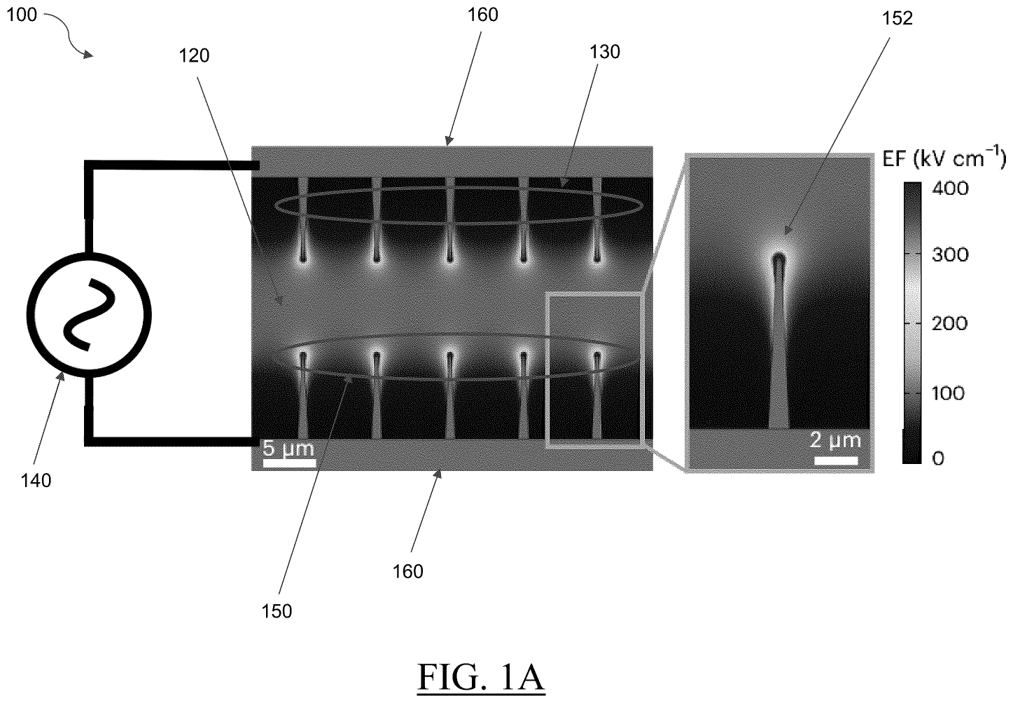

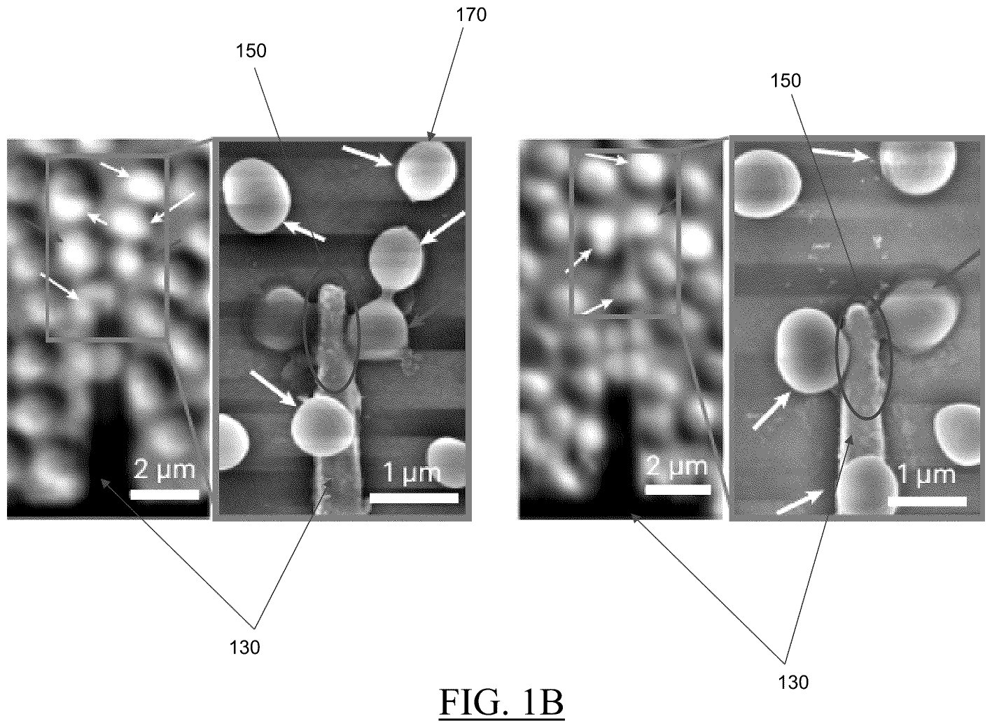

FIG. 1A is a plot of simulation results of an exemplary antimicrobial surface 120 for an antimicrobial/antifouling system 100. The antimicrobial surface 120 can include a plurality of electrodes 130 that can be configured to generate an electric field capable of inactivating microorganisms 170. The antimicrobial surface 120 can also include an insulative material that can coat at least a portion of each of the plurality of electrodes 130 to prevent shorting between the plurality of electrodes 130. The antimicrobial surface can include an external power source 140 that can be configured to supply electrical power to the plurality of electrodes 130, as illustrated in FIG. 1A. The antimicrobial surface 120 can also include one or more contact pads 160, as illustrated in FIG. 1A, that can be affixed to the plurality of electrodes 130 and can be configured to be electrically connected to the external power source 140. The antimicrobial surface 120 can be flexible, which can allow the antimicrobial surface 120 to be affixed to curved surfaces. In some embodiments, the antimicrobial surface 120 can also comprise a substrate. As known in the art, substrates can be used to provide interconnections between electrical components. With respect to the present disclosure, the substrate can provide interconnections to the plurality of electrodes 130 once fabricated. As will be appreciated, substrates can be used for a plethora of microelectronic fabrication techniques including but not limited to etching, photolithography, computer numerical control (CNC) manufacturing, and the like. With respect to the present disclosure, the substrate can be fabricated with the plurality of electrodes 130 via photolithography.

In some embodiments, the external power source 140 can be configured to provide alternating current (AC) and/or direct current (DC) electrical power to the plurality of electrodes 130 to induce the electrical field. As one skilled in the art will appreciate, the presence of the insulating material can allow the antimicrobial/antifouling system 100 to operate utilizing DC electrical power supplied by the external power source 140. In the absence of the insulating material, it can be preferable to supply the antimicrobial/antifouling system 100 with AC electrical power from the external power source 140. In some embodiments, the external power source 140 can supply DC electrical power in one or more pulse widths or with alternating polarities. The external power source 140 can be configured to provide DC electrical power that spans an approximate range of 1-500 volts (V). In some embodiments, with respect to AC electrical power, the external power source 140 can provide AC electrical power having a voltage and frequency range of 1-500 V and 10⁻³-10⁹ Hertz (Hz), respectively. The external power source 140, when supplying AC electrical power, can also be configured to supply a waveform selected from a group consisting of: sinusoidal, exponential, triangle, square, and bell. The use of various waveforms can be advantageous as different types of waveforms can modulate the strength of the electrical field generated by the plurality of electrodes 130.

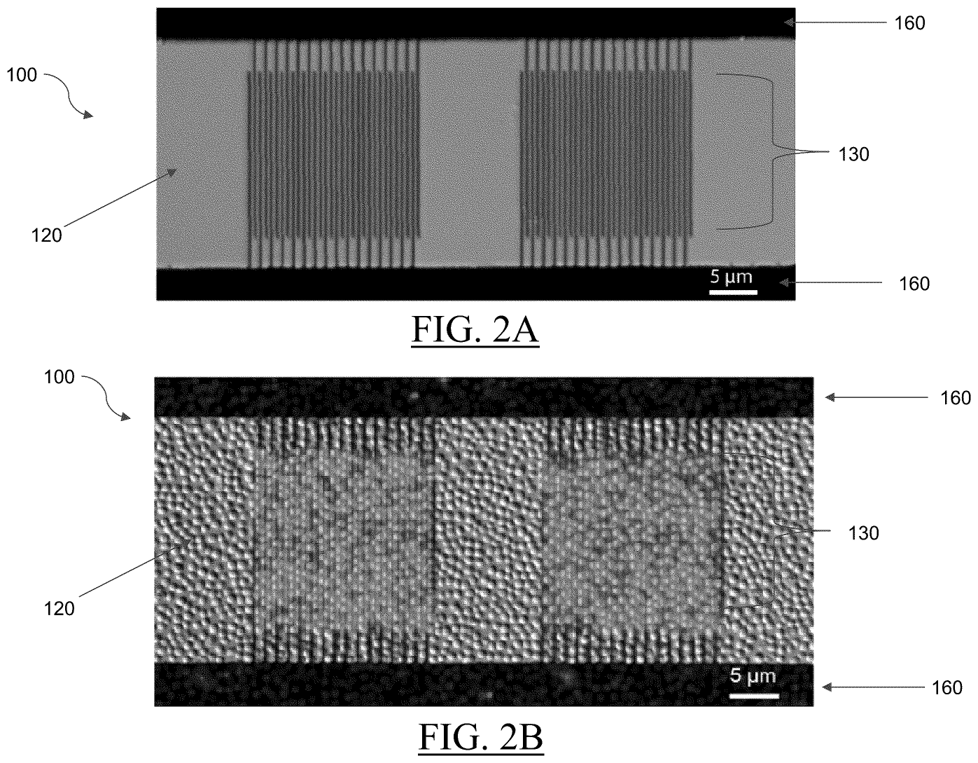

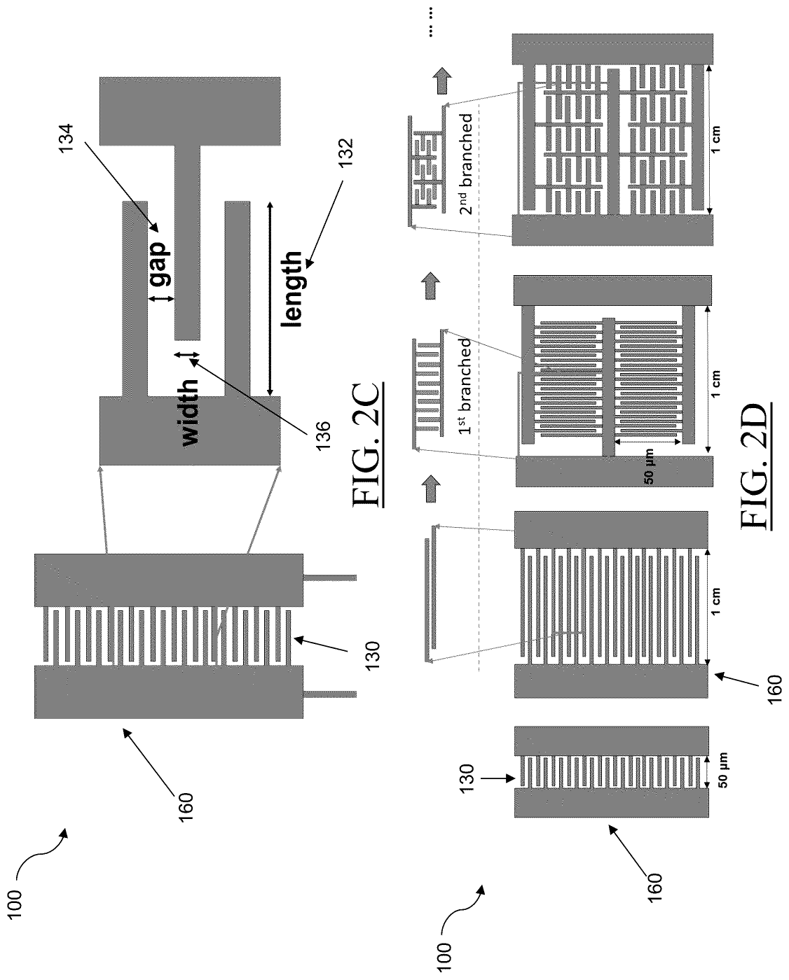

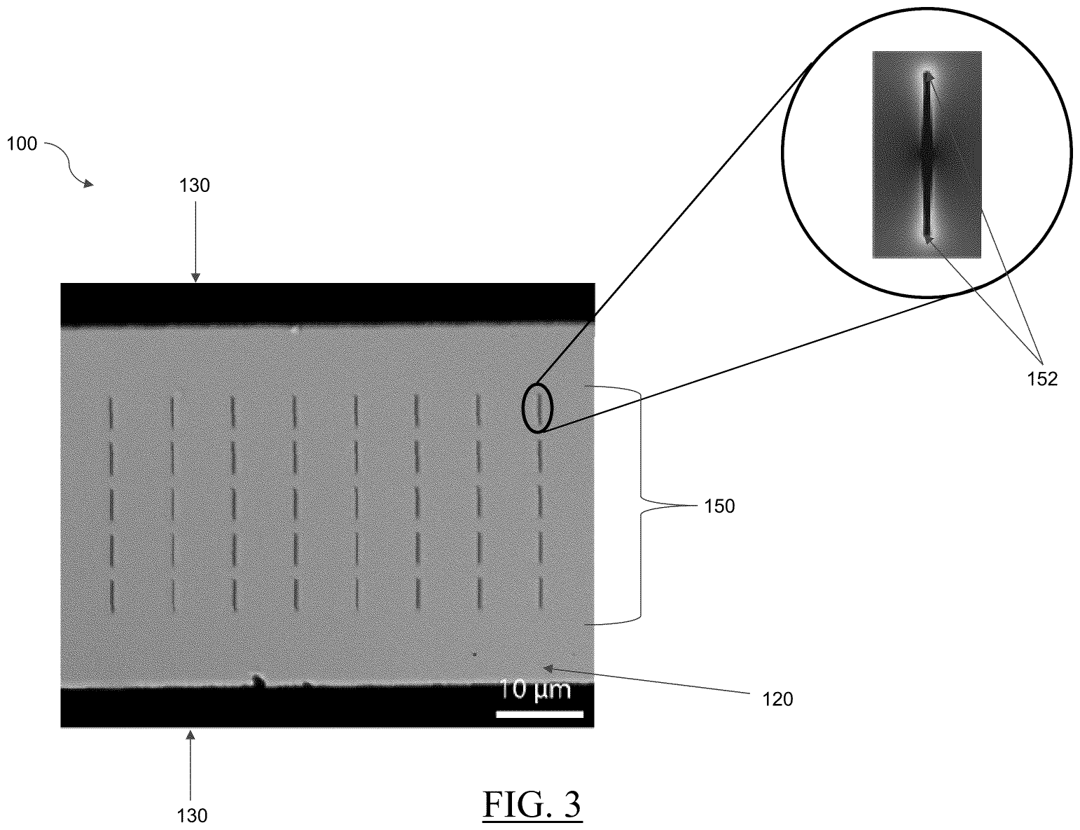

In some embodiments, the plurality of electrodes 130 can be arranged in a predetermined pattern. The predetermined pattern on the antimicrobial surface 120 can include but not limited to an interdigitated pattern, as illustrated in FIGS. 2A-2D, a plurality of nanowedges 150 between a plurality of electrodes 130, as shown in FIGS. 1A and 3, and the like. It should be appreciated, however, that other predetermined design structures can be employed on the antimicrobial surface 120 and the present disclosure is not so limited to the previously mentioned examples. In some embodiments, the predetermined design can be fabricated utilizing electronic fabrication techniques including but not limited to e-beam lithography, photolithography, lift-off method, and the like. For example, as shown in FIGS. 1A, 2A-2D, and 3, the horizontal interval between/among the plurality of electrodes 130 and/or the plurality of nanowedges 150 can be customizable. In other words, the interval of spacing horizontally and vertically between the plurality of electrodes 130 and the plurality of nanowedges 150 can be adjusted during fabrication.

Adjusting the horizontal and vertical distance between the plurality of electrodes 130 and/or the plurality of nanowedges 150 can be advantageous as it can allow adjustment of the strength of the electric field given supplied electrical energy from the external power source 140. In some embodiments, the strength of the electric field generated by the plurality of electrodes 130 can be at least approximately 1 kilovolt per centimeter (1 kV/cm). As one skilled in the art will appreciate, the threshold of approximately 1 kV is selected as it is a value high enough within an applied electric field that can damage or inactivate microorganisms 170 disposed within a biofilm on a surface or membrane. Resultantly, for the antimicrobial/antifouling system 100 to achieve a strong electric field, it can be advantageous to decrease the horizontal or vertical distance between the plurality of electrodes 130 as shown in FIGS. 2A-2D.

As mentioned previously, the antimicrobial surface 120 can comprise insulative material which can coat at least a portion of each of the plurality of electrodes 130. In some embodiments, the plurality of nanowedges 150 may also be coated with the same insulative material that can coat at least a portion of each of the plurality of electrodes 130. As one skilled in the art will appreciate, the use of the insulative material is advantageous as it can prevent “shorting” or unwanted electrical connections between the plurality of electrodes 130. In some embodiments, the insulative material can be selected from a group consisting of polymers and metal oxides. Examples of insulative material can include but not be limited to SiO₂, TiO₂, Al₂O₃, and the like. Due to the size and form factor of the plurality of electrodes 130, it can be preferable to use techniques, such as atomic layer deposition or chemical vapor deposition, in order to protect features of the plurality of electrodes 130 while simultaneously improving the durability of the antimicrobial surface 120.

FIG. 1B is a view of a side-by-side comparison between a scanning electron microscope (SEM) image and an optical microscopy image of a microorganism 170 inactivated by the antimicrobial/antifouling system 100. As indicated by the arrows in FIG. 1B, the microorganism 170 after entering the proximity of a tip 152 of at least one of the plurality of nanowedges 150 can become inactivated at least in part by the electric field generated by the plurality of electrodes 130. Based on the form factor of the plurality of nanowedges 150, in some embodiments, regions near tips 152 of each of the plurality of nanowedges 150 can be where the electric field is the strongest, as shown in FIG. 1A. In both images of FIG. 1B, it can be observed that at least some of the microorganisms 170 may experience a deformation of shape, which can be caused by damage to the cell membrane of the microorganism 170 resulting from the electric field near the tips 152 of each of the plurality of nanowedges 150.

As one skilled in the art will appreciate, the inactivation of microorganisms 170 through an electric field can be understood via principles of electroporation. Electroporation, with respect to the present disclosure, can be concisely understood as the utilization of an electrical pulse to create a temporary pore in the membrane of a microorganism 170 thus inactivating the microorganism 170. With respect to the present disclosure, application of successive pulses, which can generate an electric field via the plurality of electrodes 130 can thereby induce electroporation and thus can inactivate the microorganism 170.

FIGS. 2A-2B are microscopy views of an exemplary predetermined pattern of the plurality of electrodes 130 connected to one or more contact pads 160 of an antimicrobial/antifouling system 100 of the present disclosure. As mentioned previously, in some embodiments, the predetermined pattern of the plurality of electrodes 130 can be interdigitated as illustrated in FIGS. 2A-2B. In other words, the plurality of electrodes 130 can be at least two arrays of electrodes 130 arranged in an alternating interwoven pattern, wherein each of the at least two arrays of electrodes 130 can be connected to a respective contact pad 160 of the one or more contact pads 160. For example, the at least one of the one or more contact pads 160 can have a positive polarity and at least one of the one or more contact pads 160 can have a negative polarity. As mentioned previously, the one or more contact pads 160 can be connected to an external power source 140, which can be configured to supply electrical power to the plurality of electrodes 130 thus inducing the electric field. Given the small distance between the interdigitated pattern of the plurality of electrodes 130, the resultant electric field can be enhanced which can result in greater efficiency of the antimicrobial/antifouling system 100 disclosed herein. In some embodiments, the width of the plurality of electrodes 130 in the interdigitated pattern and the distance between each respective array can be at least approximately 10 nanometers (nm).

FIGS. 2C-2D are illustrations of exemplary configurations of a predetermined interdigitated pattern such as those shown in FIGS. 2A-2B. In some embodiments, the plurality of electrodes 130 can be fabricated on a substrate with the predetermined interdigitated patterns shown in FIGS. 2C-2D on a μm scale or a centimeter (cm) scale. As shown in FIG. 2C, features of each electrode of the plurality of electrodes 130 of the predetermined interdigitated pattern that can be adjusted can include but not be limited to a length 132, a gap 134 between each of the electrodes 130, a width 136 or thickness of each of the electrodes 130, and the like. Modulation of the design parameters can be advantageous as it can increase the strength of the electric field generated by the plurality of electrodes 130 and thereby the efficacy of the antimicrobial/antifouling system 100.

To enable scalability of design, the fabricated predetermined interdigitated patterns can be converted from a μm scale to a cm scale using different design configurations. For example, in some embodiments, the fabricated predetermined interdigitated pattern can have a “branched” interdigitated configuration such as the one shown in FIG. 2D. In other words, subsequent interdigitated patterns of electrodes 130 can “branch” or extend from one or more primary interdigitated patterns of electrodes 130. Resultantly, the one or more primary interdigitated patters of electrodes 130 can serve as a main structure and additional subsequent interdigitated patterns of electrodes 130 can branch out of the main structure. This branching configuration can be advantageous as it can allow for fabrication of predetermined interdigitated patterns with structural complexity while simultaneously providing scalability of the antimicrobial/anti-fouling system 100. For example, as shown in FIG. 2D, the predetermined interdigitated pattern may have 2 levels or “branches” of electrodes 130. In another examples, as also shown in FIG. 2D, the predetermined interdigitated pattern may have 3 levels or “branches” of electrodes. In some embodiments, design parameters for each of the plurality of electrodes 130 can include each of the electrodes 130 having a length of approximately 50 μm and each of the electrodes having approximately a 2 to 5 μm gap between each of the plurality of electrodes 130.

FIG. 3 is a microscopy view of a plurality of nanowedges 150 between a plurality of electrodes 130, including an inlet of one of the plurality of nanowedges of the antimicrobial/antifouling system 100. As shown in FIG. 3, in some embodiments, the plurality of nanowedges 150 can be suspended between the plurality of electrodes 130. In other words, the plurality of nanowedges 150 can be understood as “floating nanowedges” between the plurality of electrodes 130. The plurality of nanowedges 150 can have a high aspect ratio with a range between approximately 10 and 1,000,000. In some embodiments, the plurality of nanowedges 150 can be densely arranged, having small distances vertically and horizontally with respect to each other. As one skilled in the art will appreciate, design trade-offs can exist between densely arranging the plurality of nanowedges 150, adjusting the aspect ratio for each of the plurality of nanowedges 150, and the strength of the resultant electric field once electric energy is applied to the plurality of electrodes 130. In some embodiments, the electric field generated by the plurality of the nanowedges 150 can be concentrated at the tips 152 of the plurality of nanowedges 150. The concentration of the electric field at the tips 152 of the plurality of nanowedges 150 can enhance the strength of the electric field due to the lightning rod effect. In other words, given the geometry of the plurality of nanowedges 150 near the tips 152, the electric field strength can be enhanced due to high charge concentrations at the tips 152 of the nanowedges 150 field when a voltage is applied. In some embodiments, the strength of the electric field concentrated at the tips 152 of the plurality of nanowedges 150 can achieve values of approximately 40 kV/cm, as shown in FIGS. 1A and 3.

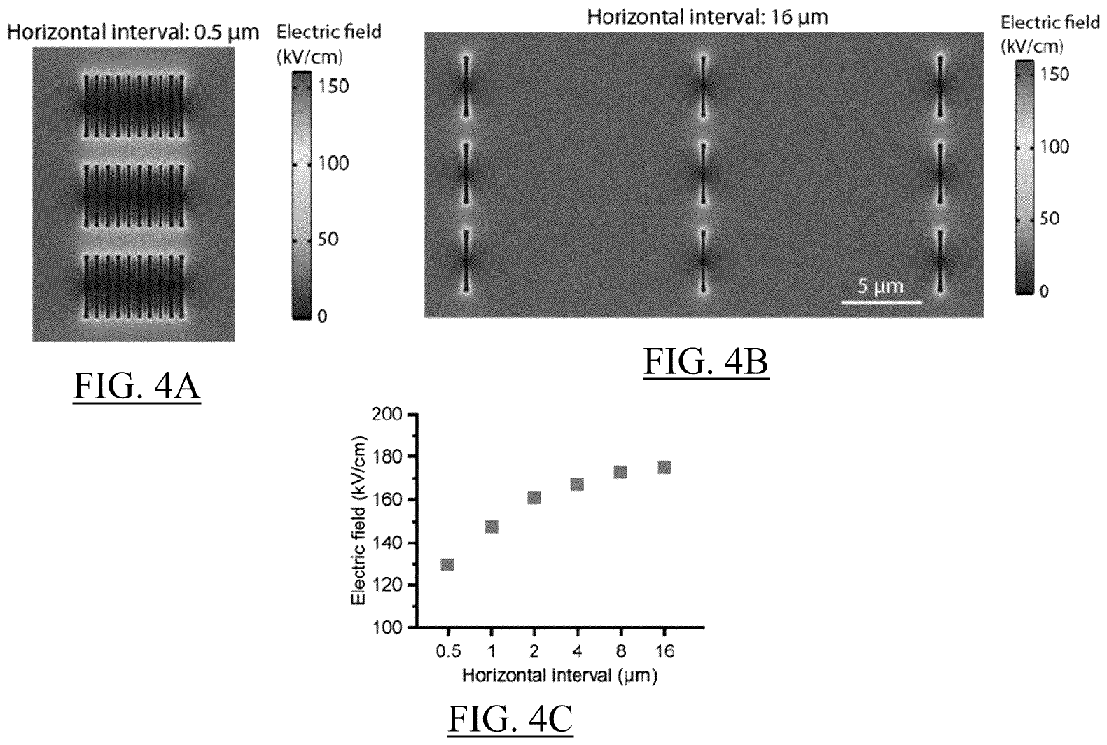

FIGS. 4A-4B are plots of simulation results for pluralities of nanowedges 150 having various horizontal intervals of spacing between each of the plurality of nanowedges 150. As observed in FIG. 4A, the plurality of nanowedges 150 can be densely arranged horizontally, having approximately 0.5 μm between each respective nanowedge 150, while maintaining a uniform vertical interval of spacing between each row of the plurality of nanowedges 150. It should be appreciated, however, that the vertical interval of spacing between each row of the plurality of nanowedges 150 can be customized and need not be uniform as observed in FIG. 4A. In contrast, as shown in FIG. 4B, the plurality of nanowedges 150 can have a larger horizontal interval of spacing with respect to each other. In some embodiments, each of the plurality of nanowedges 150 can have a horizontal interval of spacing of approximately 16 μm between each respective nanowedge 150, as shown in FIG. 4B. As also shown in both FIG. 4A-4B, locations where the electric field is strongest for the plurality of nanowedges 150 can be observed near in regions near the tips 152 of the plurality of nanowedges 150.

FIG. 4C is a plot of the relationship between the strength of an applied electric field versus horizontal intervals of spacing between each of the plurality of nanowedges 150. Referring back to FIGS. 4A-4B, horizontal intervals of spacing between each of the plurality of nanowedges 150 can be seen to be between approximately 0.5 μm and 16 μm. In contrast to the design tradeoffs of the interdigitated arrangement pattern, as illustrated in FIG. 4C, the greater the distance between each of the plurality of nanowedges 150, the greater the strength of the electric field. In some embodiments, other design parameters of the plurality of nanowedges 150 can include but not be limited to material composition, length, width, and the like. It will be appreciated that each of these design parameters can be user definable to impact the efficacy and strength of the electric field realized by the plurality of nanowedges 150.

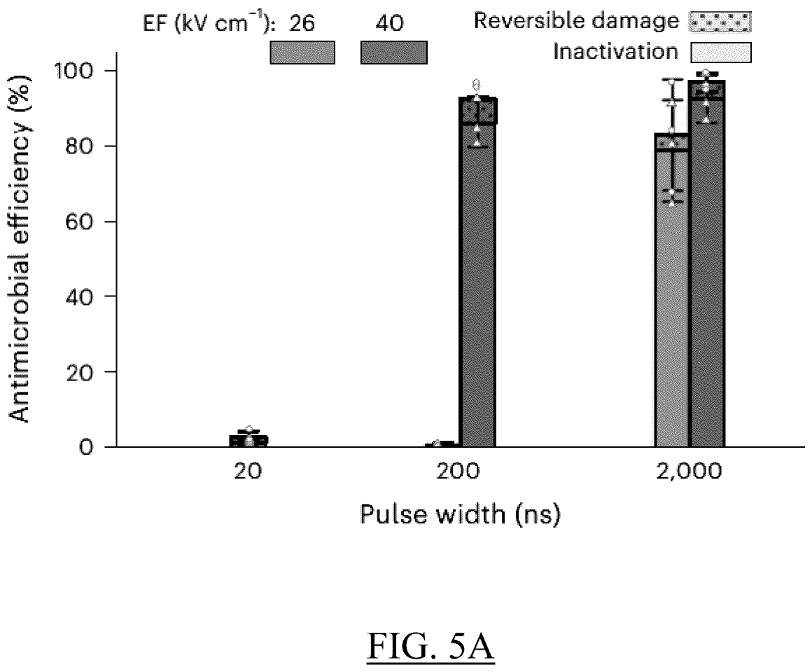

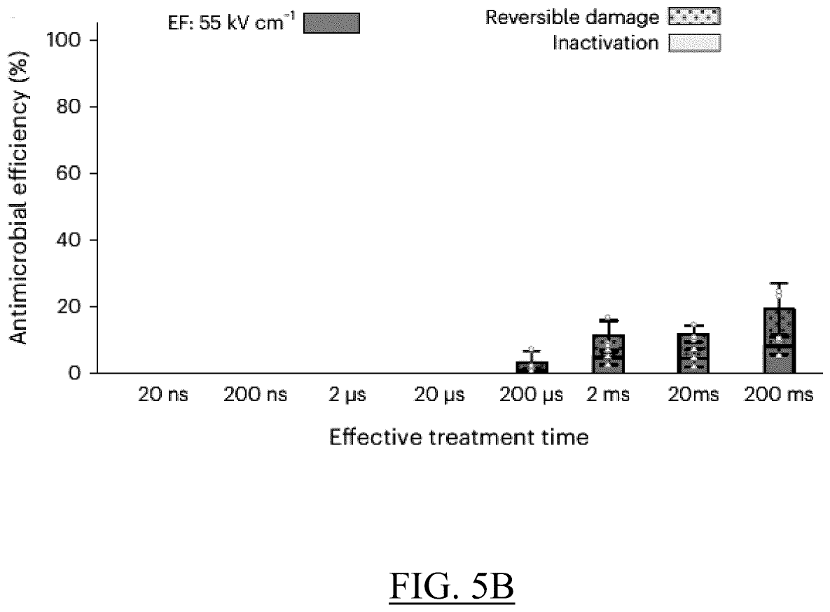

FIGS. 5A-5B are plots showing the relationship between strength and duration of the applied electric field versus efficiency of inactivating microorganisms 170 via current conventional biofilm control methods. For exemplary purposes, the conventional biofilm control method contemplated in the plots shown in FIGS. 5A-5B are conventional electric field treatment (CEFT) systems and methodologies, wherein a voltage is applied to two parallel electrode plates to induce an electric field. As shown in FIG. 5A, various pulse widths of 20, 200, and 2000 nanoseconds (ns) with electric field strengths of approximately 26 and 40 k V/cm over 20 millisecond (ms) treatment times can be measured to determine microorganism 170 inactivation efficacy. Resultantly, pulse widths of 20 ns can be observed to see minimal efficacy at both electric field strengths of approximately 26 and 40 kV/cm for CEFT. Additionally, as shown in FIG. 5A, pulse widths of 200 ns and 2000 ns for CEFT can be observed to have higher antimicrobial efficiency than pulse widths of 20 ns.

As shown in FIG. 5B, the antimicrobial efficiency of CEFT can be measured against total effective treatment time. With respect to FIG. 5B, a mathematical expression that can be used to calculate total effective treatment time can be understood as a 20 ns pulse width multiplied by the number of pulses applied during use of CEFT. In short:

Total effective treatment time=pulse width*number of pulses applied

Resultantly, it can be observed in FIG. 5B that CEFT requires higher electric field strength at longer total effective treatment times in order to reach marginal levels of antimicrobial efficiency. For example, at 20 ms of total treatment time with 20 ns pulses, CEFT can apply approximately 1,000,000 pulses at an electric field strength of 55 kV/cm, which can be observed to achieve approximately 19.4% antimicrobial efficacy. In other words, despite higher electric field strength and longer total effective treatment times at smaller pulse widths, CEFT can be observed to have suboptimal antimicrobial efficacy as shown in FIG. 5B.

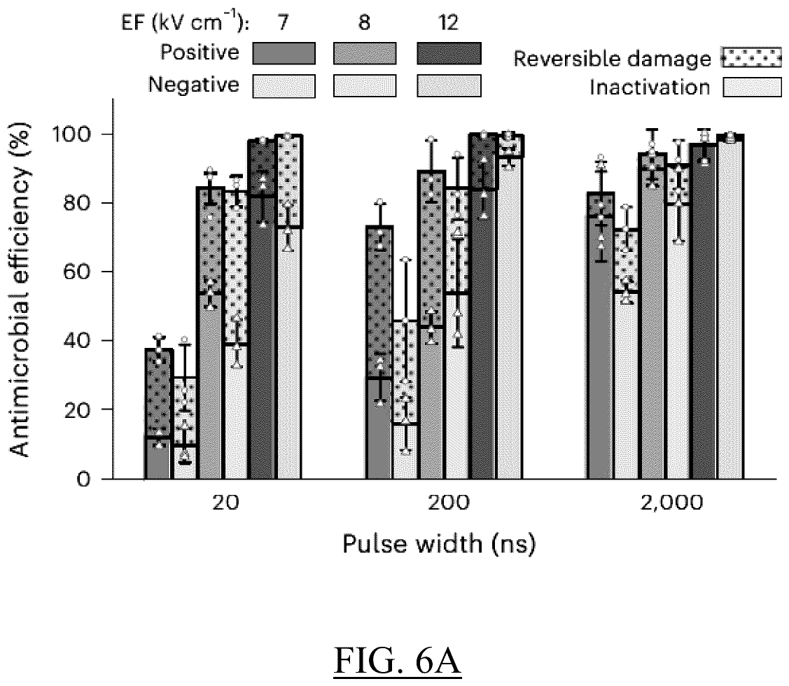

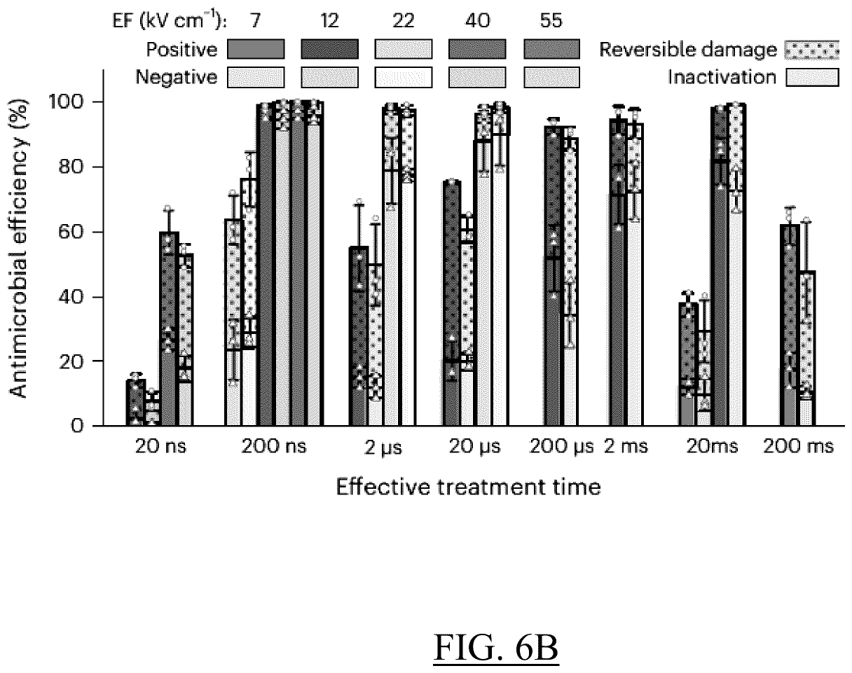

FIGS. 6A-6B are plots showing the relationship between strength and duration of the applied electric field versus efficiency of inactivating microorganisms 170 via an exemplary embodiment of the presently disclosed antimicrobial/antifouling system 100. For exemplary purposes, embodiments of the presently disclosed antimicrobial/antifouling system 100 can also be understood as locally enhanced electric field treatment (LEEFT). In other words, LEEFT can enhance the electric field generated by conventional methods, such as CEFT, by employing a plurality of nanowedges 150 with electric field strengths concentrated at the tips 152 of the plurality of nanowedges 150, which can improve antimicrobial efficiency. As shown in FIG. 6A, the electric field strengths of 7, 8, and 10 kV/cm, concentrated at the region near the tips 152 of the plurality of nanowedges 150, as shown in FIGS. 1A and 3, can achieve antimicrobial efficacy substantially greater than CEFT systems and methodologies at pulse widths of 20 ns. In short, utilizing LEEFT with electric fields concentrated at the tips 152 of the plurality of nanowedges 150 can outperform CEFT utilizing lower applied voltages, which can improve safety and efficacy.

As further evidenced in FIG. 6B, 20 ms effective treatment time of LEEFT at 20 ns pulse with an electric field strength of 7 kV/cm can result in approximate antimicrobial efficiency of approximately 37%. Based on these results, it can be shown that after an approximate 8-fold reduction in electric field strength for LEEFT, there can be higher antimicrobial efficiency than what can be observed in FIG. 5B for CEFT methodologies. Resultantly, FIGS. 6A-6B can be understood to demonstrate that LEEFT, in the small region near the tips 152 of the plurality of nanowedges 150 at lower electric field strengths, can more efficiently inactivate microorganisms 170 than current CEFT methodologies.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed:

1. A system for microorganism and/or biofilm inactivation, the system comprising:
an antimicrobial surface, the antimicrobial surface comprising
a plurality of electrodes arranged in a predetermined pattern, the plurality of electrodes configured to generate an electric field capable of inactivating microorganisms;
an insulative material coating at least a portion of each of the plurality of electrodes; and
an external power source configured to supply electrical power to the plurality of electrodes to at least in part induce the electric field via the plurality of electrodes.

2. The system of claim 1, wherein the antimicrobial surface is flexible and configured to be affixed to flat and curved surfaces.

3. The system of claim 1, wherein the antimicrobial surface further comprises one or more contact pads affixed to the plurality of electrodes, the one or more contact pads configured to be electrically connected to the external power source.

4. The system of claim 3, wherein the predetermined pattern of the plurality of electrodes is an interdigitated pattern with each of the plurality of electrodes separated by a horizontal spacing interval of at least 10 nm.

5. The system of claim 3, wherein the predetermined pattern of the plurality of electrodes is an interdigitated pattern with a vertical spacing interval between the one or more contact pads of at least 10 μm.

6. The system of claim 1, wherein the electric field generated by the plurality of electrodes is at least 1 kilovolt per centimeter (1 kV/cm).

7. The system of claim 1, further comprising one or more nanowedges distributed between the plurality of electrodes, each of the one or more nanowedges having a predetermined horizontal or vertical spacing interval, with respect to each other.

8. The system of claim 7, wherein the one or more nanowedges are affixed to the plurality of electrodes, each of the one or more nanowedges having the predetermined horizontal or vertical spacing interval, with respect to each other.

9. The system of claim 1, wherein the insulative material is configured to electrically protect the plurality of electrodes against short circuiting.

10. The system of claim 1, wherein the insulative material comprises a material selected from the group consisting of polymers and metal oxides.

11. The system of claim 1, wherein the external power source is further configured to provide alternating current (AC) electrical power to the plurality of electrodes to induce the electrical field.

12. The system of claim 11 wherein the external power source is configured to provide AC electrical power with a voltage range of 1-500 volts (V) and a corresponding frequency range of 10⁻³-10⁹ Hertz (Hz).

13. The system of claim 12, wherein the external power source is configured to provide AC electrical power with a waveform selected from a group consisting of: sinusoidal, exponential, triangle, square, and bell.

14. The system of claim 1, wherein the external power source is further configured to provide direct current (DC) electrical power to the plurality of electrodes to induce the electrical field.

15. The system of claim 14, wherein the external power source is configured to provide DC electrical power with a voltage range between 1-500 volts (V).

16. The system of claim 15, wherein the external power source is configured to provide DC electrical power periodically in alternating polarities.

17. The system of claim 15, wherein the external power source is configured to provide DC electrical power with one or more predetermined pulse widths.

18. A system comprising:
a substrate;
a plurality of electrodes distributed on the substrate; and
a power source configured to provide power to the plurality of electrodes, such that the plurality of electrodes produce an electric field on at least a portion of the substrate sufficient to achieve an antimicrobial or antifouling result.

19. The system of claim 1, further comprising a plurality of nanowedges distributed between the plurality of electrodes, each of the plurality of nanowedges having an aspect ratio between 10 and 1,000,000.

20. The system of claim 18, wherein the plurality of electrodes are interdigitated.
